(19) United States
(12) Patent Application Publication
(10) Pub. No.: US 2019/0275703 A1
(43) Pub. Date: Sep. 12, 2019

(54) GRANULATE PRODUCTION WITH ROUNDED PARTICLES FOR MANUFACTURING IMPLANTS OR TOOL MANUFACTURING

(71) Applicant: Karl Leibinger Medizintechnik GmbH & Co. KG, Mühlheim (DE)

(72) Inventors: Adem AKSU, Villingen-Schwenningen (DE); Frank REINAUER, Emmingen-Liptingen (DE); Tobias WOLFRAM, Dreieich (DE)

(21) Appl. No.: 16/304,617

(22) PCT Filed: Jan. 17, 2017
(86) PCT No.: PCT/EP2017/050894

§ 371 Date: Nov. 26, 2018

(30) Foreign Application Priority Data
Jun. 7, 2016 (DE) 10 2016 110 501.5

(51) Int. Cl. B29B 9/12; B29B 9/02; B29B 9/08; B33Y 70/00; A61F 2/30; A61L 27/18; A61L 27/56

(52) U.S. Cl. CPC B29B 9/12; B29B 9/02; B29B 9/08; B33Y 70/00; B29K 2023/0683; A61L 27/18; A61L 27/56; B29B 2009/125; A61F 2/30

(57) ABSTRACT

The invention relates to a method for producing a plastic object (1) for surgical use, comprising the following steps: a) providing a plastic powder (2); b) heating and pressing the plastic powder (2) thus forming at least one intermediate piece (3); c) mechanically comminuting the at least one intermediate piece (3) to form a granulate (4); and d) joining the granulate (4) to form an integral base body (6). The invention also relates to an implant or to an auxiliary means having at least one base body (6) comprising a UHMWPE material.

The invention relates to a method for producing an object/a utensil, especially an implant or a surgical tool/auxiliary means comprising plastic material, preferably consisting of plastic material and provided for surgical use. The invention also relates to an implant, an auxiliary means and a tool.

It is already basically known from the state of the art to use UHMWPE materials (“ultra-high molecular weight polyethylene materials) for producing implants. For example, U.S. Pat. No. 6,641,617 B1 discloses a medical implant for use within a body, wherein said implant is formed of said UHMWPE material.

It has turned out to be a drawback, however, in the already known methods that the manufacture of the medical objects used in surgery, for example of implants, frequently is relatively complex. This is especially due to the fact that starting plastic material has to show a specific shape (grain size, grain shape etc.) and simultaneously relatively high viscosity. Therefore, in prior art, for the manufacture of implants of plastic material predominantly chemical process steps are resorted to by making use of various solvents.

It is the object of the present invention to eliminate the drawbacks known from prior art and, especially, to make available a method for manufacturing a medical object of plastic material the expenditure of which is to be significantly reduced.

According to the invention, this is achieved by a method according to the first claim, wherein for manufacturing an object consisting of plastic material and being provided for surgical use, especially an implant or a surgical/medical aid such as an (operating) tool, at least the following steps are realized:
a) providing (a particular amount (volume or weight)) of a (free flowing) plastic powder;
b) heating and pressing the plastic powder thus forming at least one intermediate piece/to form at least one intermediate piece;
c) mechanically comminuting the at least one intermediate piece to form a granulate (preferably having a predetermined grain size and/or grain shape); and
d) joining the granulate to form a base body.

The object is further achieved by a (preferably plate-shaped) implant having at least one porous base body comprising a UHMWPE material.

By the afore-mentioned method steps, the object is provided largely without any chemical reaction steps, such as by solutions, and predominantly or completely by mechanical and, resp., physical working steps. By pressing the plastic powder to form intermediate pieces as well as by the subsequent mechanical comminuting defined and uniform particles can be used as granulate so that a preferably reproducible manufacturing method is realized. Especially in this way the intended porosity of the object to be manufactured, preferably of the implant, can be specifically adjusted. As reactants usually to be degraded in a biologically complicated manner are most largely dispensed with, the environmental pollution is substantially reduced.

Further advantageous embodiments are claimed in the subclaims and will hereinafter be explained in detail.

The object provided for surgical use advantageously is an implant which further preferred serves for osteosynthesis or fracture repair and/or for forming a (patient-specific) individual implant. In further configurations it is also useful when the object provided for surgical use is in the form of an operation aid, such as an (operating) tool for medical use during an operation.

The plastic powder preferably consists of one single plastic material. The manufacturing method then can be especially easily reproduced.

Accordingly, it is especially advantageous when the plastic powder consists of a thermoplastic material, of preference polyethylene (PE), especially preferred ultra-high molecular polyethylene (UHMWPE), further preferred high-density polyethylene (HOPE), even further preferred polypropylene (PP) such as a polypropylene fumarate (PPF), or even further preferred polyaryletherketone (PAEK), especially polyetheretherketone (PEEK). This helps to adjust the object, preferably an implant, optimally in terms of material to the respective fields of use.

It is also of advantage when the plastic powder consists of thermosetting resin material, especially preferred of biologically degradable biocompatible thermosetting resin material, preferably (thermosetting) polyurethane (PUR), further preferred polyacrylate or epoxy resin. In that case, too, especially efficient implants as an object can be manufactured.

In addition, it is of advantage especially for manufacturing an auxiliary means such as a tool, for the object when the plastic powder is made from elastomeric material, of preference an (elastomeric) polyurethane (PUR), further preferred a silicone material, especially preferred a polysulfide.

However, it is also of advantage when the plastic powder consists of different plastic materials, i.e. of different thermoplastic, thermosetting and/or elastomeric materials. Here it is especially preferred when the plastic powder consists of UHMWPE containing HOPE and/or PE admixtures. Such powder mixture is especially suited for manufacturing objects as implants. Another admixture of PP, PPF, PAEK, such as PEEK, (elastomeric and/or thermosetting) PUR, polyacrylate and/or epoxy resin to said UHMWPE-HDPE-PE mixture entails more flexible use of the object.

It is of further advantage when the base body has a porous structure. Said porous structure is especially favorable to an object in the form of a medical implant being accepted within/growing into the body of the respective mammal.

In this context, it is moreover useful when the porous structure is an open-cell or closed-cell/open-pore or closed-pore structure, i.e. has an interconnecting and/or non-interconnecting form. On the one hand, an open-pore structure helps to promote ingrowing of the object in the body of the mammal, on the other hand a closed-pore structure helps to further increase the strength.

It is especially advantageous in this context when the base body has such porosity that the pore sizes range from 10 μm to 450 μm, further preferred are less than 300 μm/range from 10 μm to 300 μm, especially preferred range from 200 μm to 300 μm. It is also advantageous when the base body has such porosity that the pore sizes range from 500 μm to 850 μm. In this way, the object is further optimized for medical use.

It is of further advantage when the base body has a porosity between 30 and 45%, further preferred between 50 and 60%, especially preferred of more than 80%. This renders the base body especially efficient.

In this context, it is moreover advantageous when at least one further additive, preferably a pro-osteosynthetic additive, is added to the plastic powder (prior to carrying out step a) or d)). Said additive preferably is hydroxy apatite (HAP), calcium carbonate (CaCO₃), magnesium (Mg), iron (Fe), strontium (Sr), alpha- or beta-tricalcium phosphate (alpha/beta TCP), bioglass® particles/particles of bioactive glass, polyester material such as PDLLA, PLGA, PLA, PGA, chitosan fibers or a chitosan particle. In this way, especially biocompatible as well as stable objects can be produced.

When the base body is moreover hydrophilized, the object is further optimized as an implant for its use within a human body.

In addition, it is of advantage when the method steps a) through d) are carried out in time succession. This renders the method especially efficient.

If the plastic powder has a grain size between about 20 μm or about 50 μm and about 900 μm, preferably between about 300 μm and about 600 μm, further preferred about 500 μm±100 μm, the manufacture of the intermediate pieces can be easily realized.

It is especially advantageous when the at least one intermediate piece is plate-shaped, has (at least in portions or completely) a porous material structure and/or is formed (at least in portions or completely) of solid material. In this way, the intermediate piece is favorably prepared as to its configuration for the subsequent comminution.

Moreover, it is useful when step c) includes a first partial step c1) in which the at least one intermediate piece is pre-comminuted, preferably by machining, cutting and/or punching, into single pieces, and/or includes a second partial step c2) in which the single pieces are (further) comminuted, preferably by milling, to form granulate/pellets. This helps to produce the granulate especially precisely as to shape and size.

It is further advantageous when in the second partial step c2) milling is performed by means of a rotor mill, a rotor and/or a screen of the mill being preferably configured as a Conidur® plate or a plate having plural holes such as round holes. Thus, a final shape of the granulate can be realized especially skillfully. However, it is recommendable to use liquid nitrogen, as then adhesion in the screen or in the rotor is avoided. Rotor shapes in the form of a beater and a turbo rotor in combination with a round-hole screen and/or a Conidur screen have especially proven themselves, wherein they have a rather triangular to semi-elliptic opening as compared to the slit-hole sheets.

In this context, it is particularly advantageous when the shape of the particles of the granulate is (preferably uniformly) round, oval, triangular and/or rectangular. Especially, it is of advantage when each of the particles has a rounded surface, i.e. rounded edges. Thus, the object is manufactured to be especially durable.

It is also useful with respect to the second partial step c2) when the particles of the granulate show a grain size between 20 μm and 2000 μm after carrying out the second partial step c2). Said grain sizes are especially suited for use of the object as a medical implant.

If step d) comprises sintering of the granulate, preferably porous sintering and/or selective laser-sintering, i.e. if step d) is carried out as a sintering operation, the object can be perfectly manufactured by automation.

In this event, the sintering operation per se is preferably carried out in a nitrogen and/or argon atmosphere or in vacuum/under vacuum. Also, moreover a membrane may be used during sintering, thus allowing the generation of the atmosphere/the vacuum to be realized especially efficiently and a particular elasticity to be given. This renders the manufacturing method even more efficient.

After sintering or during sintering, also additional pressing or additional leaching of different material components/of the plastic material of the base body can be carried out, thus causing the porosity of the object to change locally or in total. In this context, it is also especially advantageous when during sintering a biodegradable material such as HAP, CaCO₃, alpha/beta/x-TCP etc. is filled in and is appropriately cross-linked with the granulate of the plastic material. In this manner, the mechanical properties of the object can be adjusted especially skillfully. The particles may take different shapes, as afore-mentioned already, wherein preferably they take a rounded shape so as to provide optimum energy input during sintering.

It is further advantageous when on the base body, preferably following step d), in step e) a sterilizing radiation of the base body is carried out so that the plastic material (additionally) cross-links. In this way the stability of the object is further improved.

In this context, it is especially advantageous when gamma sterilizing radiation at preferably 10 to 45 kGy, further preferred at about 25 kGy (corresponding to 2.5 billion rad), sterilizing radiation/vaporization with ETO gas, e-beam sterilization or plasma sterilization is performed. Thus, also the radiation can be realized especially efficiently by automation.

If moreover the base body is cleaned, preferably following the steps d) and e) or between the steps d) and e), in a step f), the quality of the object/base body is further improved.

It is of advantage when the base body is cleaned, especially by means of snow blasting, for example using frozen CO₂.

It is especially expedient when cleaning of the base body involves ultrasonic bath cleaning and/or surface treatment, e.g. a surface treatment such as snow blasting by means of technologies based on CO₂ or a thermal surface finishing. The ultrasonic bath cleaning is carried out, further preferred, by means of ethanol or isopropanol. Thus, an especially high degree of purification is obtained.

It is also advantageous when, preferably following the steps d), e) and/or f), in a further step (preferably step g)) a (preferably thermal) surface treatment of the base body is carried out. All particles of the base body thus can be especially permanently fixed.

If the surface treatment comprises a plasma/low-pressure plasma surface treatment (in the form of thermal finishing treatment), better ingrowing behavior is achieved. Especially an increase in strength of UHMWPE, HOPE and PP implants as well as fixing of the remaining particles on the surface is achieved.

If the surface treatment comprises, additionally or alternatively to the plasma/low-pressure plasma surface treatment, hot air temperature treatment, preferably by a hot air blower, there is further given the option of subsequent intraoperative shaping by heat treatment so that again an increase in strength of UHMWPE, HOPE and PP implants by thermal finishing treatment and fixing of the remaining particles on the surface is achieved.

If the surface treatment, preferably in addition to the hot air/hot air temperature treatment, comprises explosion deburring on a specific plastic system or the like, again there is given the option of subsequent interoperative shaping by heat treatment so that an increase in strength of UHMWPE, HOPE and PP implants by thermal finishing treatment, such as hot air blower, and fixing of the remaining particles on the surface (thus interconnecting increase in strength) is achieved.

If, furthermore, the surface treatment in addition or as an alternative comprises a treatment of the surface of the base body with supercritical CO₂, another option of subsequent intraoperative shaping by heat treatment is provided and the increase in strength of UHMWPE, HOPE and PP implants by thermal finishing treatment as well as fixing of the remaining particles on the surface, i.e. an interconnecting increase in strength, is further evolved.

If the surface treatment in addition or as an alternative comprises a treatment of the surface of the base body with infrared light by infrared radiators, another option of subsequent intraoperative shaping by heat treatment is provided and the increase in strength of UHMWPE, HOPE and PP implants by thermal finishing treatment as well as fixing of the remaining particles on the surface, i.e. an interconnecting increase in strength, is further evolved.

Moreover, it is advantageous if the surface treatment additionally or alternatively comprises flame treatment of the base body, then a further option of subsequent intraoperative shaping by heat treatment is provided and the increase in strength of UHMWPE, HOPE and PP implants by thermal finishing treatment as well as fixing of the remaining particles on the surface, i.e. an interconnecting increase in strength, is further evolved.

If the surface treatment additionally or alternatively comprises heat treatment in a heating furnace, another option of subsequent intraoperative shaping by heat treatment is provided and the increase in strength of UHMWPE, HOPE and PP implants by thermal finishing treatment as well as fixing of the remaining particles on the surface, i.e. interconnecting increase in strength, is further evolved.

In this context, it is especially advantageous when the surface treatment, especially the hot air treatment, the flame treatment and/or the plasma surface treatment, is carried out by means of a robot arm. This helps to provide another option of subsequent intraoperative shaping by heat treatment and to further evolve the increase in strength of UHMWPE, HDPE and PP implants by thermal finishing treatment as well as fixing of the remaining particles on the surface, i.e. interconnecting increase in strength.

Basically, it is also referred to the fact that after carrying out the method steps of the independent claim 1, i.e. the steps a) through d), a base body already realizes a complete object such as the implant. The method steps e) through g) additionally carried out in the dependent claims further develop the base body and thus contribute to the finally obtained object being improved even more efficiently for use within a body of a mammal. The method steps e) through g) may be carried out jointly or independently of each other in addition to the steps a) through d).

When configuring the object as an implant, the base body is either adapted, when being inserted during operation, in the usual way, as to its shape to the patient-specific geometry of the bones and cartilages, but it may also exhibit the final patient-specific shape already during sintering (immediately in the wake of step d)). Said shape then in the latter case is detected by means of a scanning process of the patient's bone part concerned and is configured in the sintering step.

The invention also relates to an implant or an auxiliary means comprising at least one base body including UHMWPE material. A template or a tool qualifies as an auxiliary means. In this variant, the base body is non-porous/closed, whereas in the variant as an implant it has a porous design.

A preferred method according to the invention for manufacturing an object made from plastic/plastic material and provided for surgical use shall be described in detail hereinafter by way of a figure in an example configuration, wherein

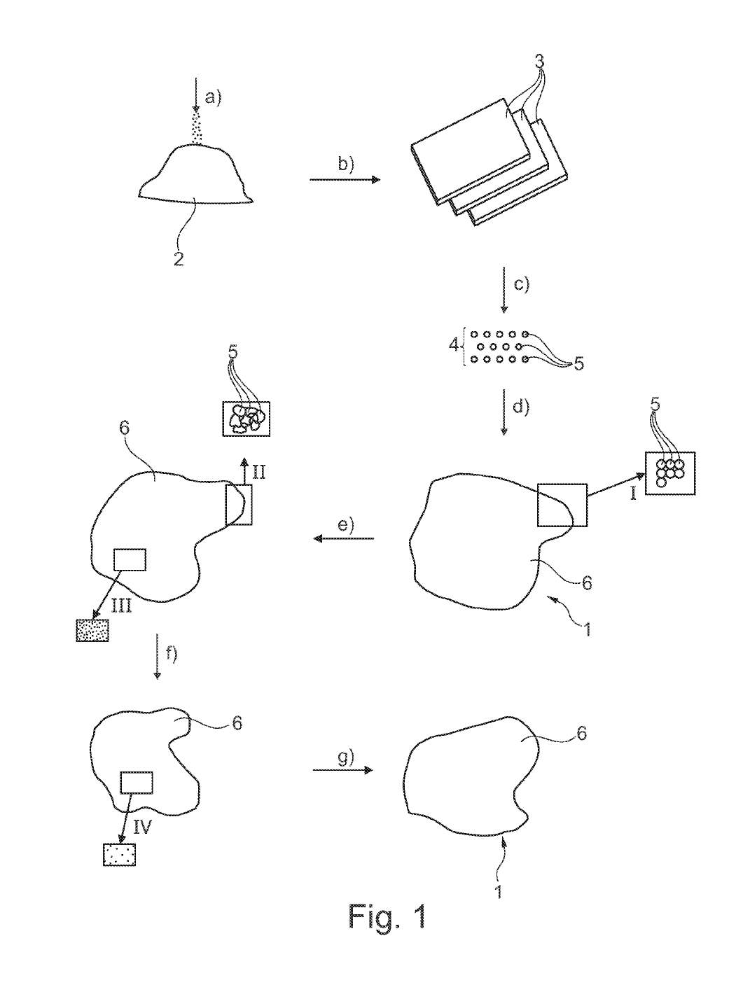

FIG. 1 shows a schematic view of the manufacturing method set forth in an example configuration according to the invention,

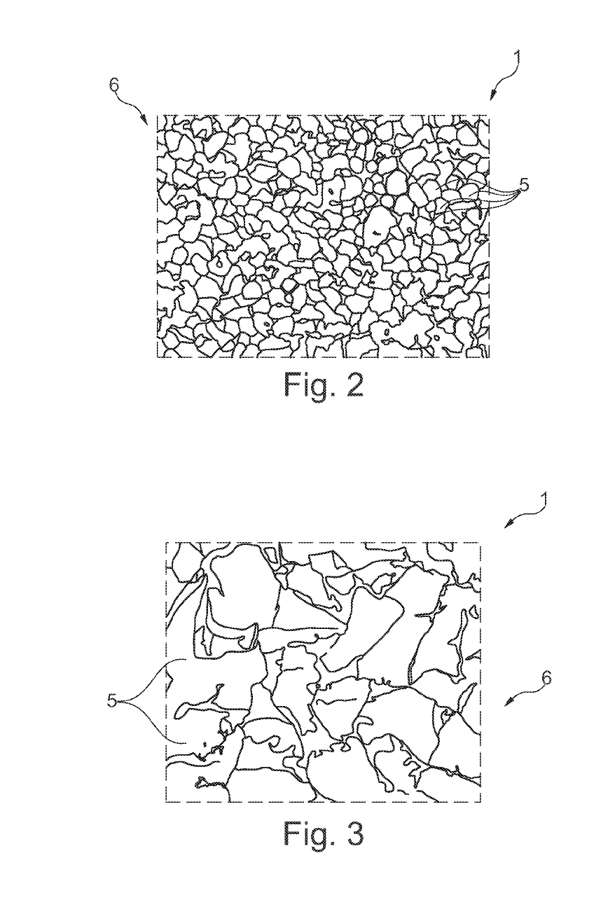

FIG. 2 shows a microscopic detailed sectional view of a section across a finished base body of an object forming an implant, as it is manufactured according to the manufacturing method set forth in FIG. 1, wherein especially the shape of the granulate used in the form of balls is evident,

FIG. 3 shows a microscopically detailed sectional view of a section across a finished base body of an object forming an implant, as it is manufactured according to a manufacturing method set forth in a second example configuration, wherein said manufacturing method differs from the manufacturing method according to FIGS. 1 and 2 by the use of polygonal particles of the granulate, and

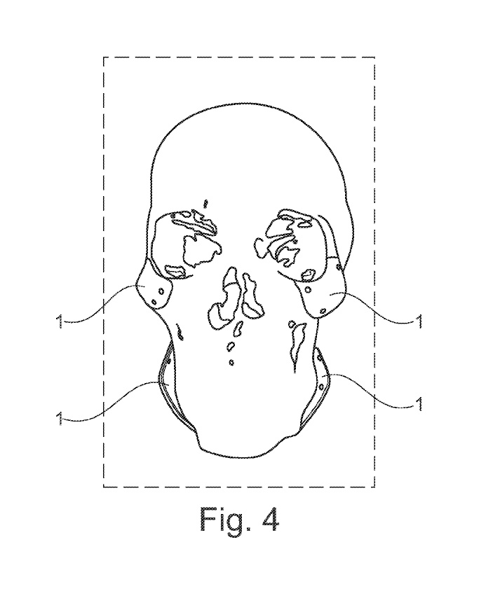

FIG. 4 shows a perspective view of a human skull for illustrating the possible attaching areas of the manufactured object/implant.

The figures are merely schematic and serve exclusively for the comprehension of the invention. Like elements are provided with like reference numerals.

In FIG. 1 a preferred manufacturing method according to the invention as set forth in a first example embodiment is clearly evident. For manufacturing an ultimately finished base body 6 which forms an object 1 provided for surgical use, i.e. a medical implant, in this method the method steps a) through g), marked by arrows, are carried out in time succession. For manufacturing the base body 6, at first the method steps a) through d) have to be carried out. As in the two example configurations described in the following the object 1 is in the form of an implant, hereinafter the implant as the object is provided with reference numeral 1. As an alternative to the manufacture of the implant 1, in further configurations also different objects, especially auxiliary means for an operation such as surgical tools are manufactured by said manufacturing method.

As is evident from FIG. 1, initially a plastic powder 2 in the form of an UHMWPE powder 2 is provided (arrow a)), wherein said plastic powder 2 has a grain size/average grain size of less than 300 μm.

The free-flowing plastic powder 2 immediately thereafter is pressed, marked by arrow b), by means of a sinter-like process. This results in one-piece/coherent intermediate pieces 3. Especially, the intermediate pieces 3 are obtained by pressing with simultaneous heating of the plastic powder 2, the intermediate pieces 3 finally forming rectangular plates. The temperature of the intermediate pieces 3 during said sintering/primary forming of the intermediate pieces 3 is always below the disintegrating temperature of the plastic powder 2 used (in plural plastic materials below the disintegrating temperature of the lowest-melting material component of the plastic powder 2 used). Of preference, for producing the respective intermediate piece 3 a female mold is provided into which the plastic powder 2 is initially filled and which is subsequently heated as well as compressed, with a compacting force being applied, so that a solid structure in the form of the intermediate pieces 3 is formed.

Following the manufacture of the intermediate pieces 3, according to arrow c) each intermediate piece 3 is comminuted again in a defined manner. The intermediate pieces 3 are comminuted into plural particles 5 while forming a granulate 4. The particles 5 have a substantially uniform shape which is brought about by the concrete execution of the mechanical comminution. In this example configuration, round particles 5 in the form of spherical particles 5 or of particles 5 being oval in cross-section are produced.

The method step c) is subdivided into two partial steps not shown in detail here for the sake of clarity. In a first partial step (referred to as first partial step c1) the at least one intermediate piece 3 is pre-comminuted by cutting so that a plurality of sharp-edged single pieces is produced in turn from one intermediate piece 3. Alternatively, it is also considered in further example configurations to produce said single pieces by machining, such as milling or turning, and/or by punching rather than by cutting or in addition to cutting.

Following the first partial step c1), the plural single pieces are mechanically further comminuted, viz. ground, in a second partial step (referred to as second partial step c)). The single pieces are ground until the uniform granulate 4, i.e. especially uniform as to size and shape, forms from a plurality of particles 5. The grinding process is preferably realized by means of a rotor mill, wherein a rotor moves relative to an area that is stationary/fixed to the housing, viz. a screen, and the single pieces disposed therebetween are comminuted due to the mechanical shear forces. The rotor and the screen in that case include plural holes which already predetermine the circumferential geometry of the finished granulate 4. Since here round particles 5 are formed, the holes/through-holes equally take a round shape. By pressing the respective single pieces through the holes, the round shape is imparted to the particles 5.

According to arrow d), then joining of the granulate 4 set as to its form will follow to form the one-piece base body 6. In this example configuration, a sintering operation, viz. a selective laser-sintering operation, will serve for joining. As an alternative, it is also possible, however, to make use of different sintering techniques, for example porous sintering or even different joining techniques, e.g. adhesive joining techniques such as welding.

After step d), the base body 6 consists of a coherent stable plastic material in the form of the UHMWPE which was present before in powdered form. As is evident from the partial representation of the schematic view according to FIG. 1 between the arrows d) and e), a substantially plate-shaped implant 1 of any configuration is already pre-shaped in the form of said base body 6. In said base body 6 the individual, previously free-flowing granulate particles 5 are adhesively tightly joined (detailed representation “I”). The base body 6 in this process exhibits substantially the finished shape of the implant 1 to be manufactured already after carrying out step d). Accordingly, the implant 1 is typically configured as an implant 1 for osteosynthesis and, resp., fracture repair, e.g. as a cranial implant. Sintering is carried out such that the implant/the base body 6 has a porous, preferably open-pore structure. Alternatively, also closed-pore structures may be realized.

In addition to the steps a) through d) which already serve for completely configuring the implant 1/base body 6, in the example configuration according to FIG. 1 the steps e) through g) are further realized. By step e) the base body 6 is further exposed, subsequent to step d), to a radiation operation, viz. to a sterilizing radiation. Said sterilizing radiation serves for additional cross-linking of the UHMWPE material, which is evident from the partial representation following arrow e) of FIG. 1 by means of a detailed cutout “II”. Accordingly, the individual particles 5 nestle even more closely to each other and, resp., enlarge their mutual contact faces.

After the sterilizing radiation according to the method step f), the base body 6 is cleaned, which is visible between the partial representations before and after the arrow f) by the detailed representations “Ill” and “IV” of the surface.

After cleaning the surface, by step g) a thermal surface finishing of the base body 6 is carried out. Finally, this results in the implant 1 finished in the wake of step g) according to the preferred example configuration.

In FIG. 2, a microscopic detailed representation of a section across the finished implant 1 from FIG. 1 is illustrated once again in detail. Here especially the round/oval cross-sectional shape of the individual particles 5 is visible.

In combination with FIG. 3 it is also possible, however, to basically provide shapes other than said round shape. In FIG. 3 showing a cross-section of a different implant 1, the particles 5 take a polygonal shape. An implant 1 of such polygonal design of its particles 5 would be feasible by a method similar to the one shown in FIG. 1, wherein merely the grinding operation according to step c2) would have to be adapted. Instead of round holes in the rotor and in the screen, angular through-holes would have to be provided. The latter may as well vary in size so that finally the particles 5 according to FIG. 3 are designed to be somewhat larger than those shown in FIG. 2.

The finished implant 1 may be used, for example, at the cranial bone or in the jaw area, as is evident from FIG. 4, or in similar areas of the mammal. Also, the implant 1/the base body 6 may be manufactured in accordance with specific geometrical data of a patient. For this purpose, it is possible to design the appropriate sintering mold already as a patient-specific female mold and thus to produce already the finished shape of the implant 1 according to step d) and, resp., according to step g). As an alternative to this, it is also possible to geometrically adapt the finished base body 6 in size by bending or cutting immediately during operation.

In further configurations it is also possible to manufacture the base body 6 from materials other than the selected UHMWPE, such as PE, PP or HOPE. Basically, also other thermoplastic materials, thermosetting and/or elastorneric resins are suited for manufacture. Also, material mixtures such as mixtures of UHMWPE, PP, PE and/or HOPE may be chosen for manufacture.

### LIST OF REFERENCE NUMERALS

1 object/implant

2 plastic powder

3 intermediate piece

4 granulate

5 particle

6 base body

1. A method for producing an object (1) comprising plastic material and being provided for surgical use, the method comprising at least the following steps:
a) providing a plastic powder (2);
b) heating and pressing the plastic powder (1) thus forming at least one intermediate piece (3);
c) mechanically comminuting the at least one intermediate piece (3) to form a granulate (4); and
d) joining the granulate (4) to form an integral base body (6).

2. The method according to claim 1, characterized in that the base body (6) has a porous structure.

3. The method according to claim 2, characterized in that the porous structure is an open-cell or closed-cell structure.

4. The method according to any one of the claims 1 to 3, characterized in that the plastic powder has a grain size between 20 μm and 900 μm.

5. The method according to any one of the claims 1 to 4, characterized in that the at least one intermediate piece (3) is plate-shaped, has a porous material structure and/or is formed of solid material.

6. The method according to any one of the claims 1 to 5, characterized in that step c) includes a first partial step c1) in which the at least one intermediate piece (3) is pre-comminuted to form single pieces and a second partial step c2) in which the single pieces are further comminuted to form granulate (4), or that in step c) a granulate (4) is produced directly from the intermediate pieces (3).

7. The method according to claim 6, characterized in that, after carrying out the second partial step c2), plural particles (5) forming the granulate (4) have a grain size between 20 μm and 2000 μm.

8. The method according to any one of the claims 1 to 7, characterized in that step d) comprises sintering of the granulate (4), preferably porous sintering and/or selective laser-sintering (SLS process).

9. The method according to any one of the claims 1 to 9, characterized in that a surface treatment is carried out on the base body (6).

10. An implant or auxiliary means having at least one base body comprising UHMWPE material.
